(19) United States
(12) Patent Application Publication
(10) Pub. No.: US 2026/0347804 A1
(43) Pub. Date: Nov. 5, 2026

(54) PFU DNA POLYMERASE MUTANT AND APPLICATION THEREOF IN PCR

(71) Applicant: Innovation Academy for Precision Measurement Science and Technology, Chinese Academy of Sciences, Wuhan (CN)

(72) Inventors: Lichun HE, Wuhan (CN); Yihao CHEN, Wuhan (CN); Shouzhen ZHAO, Wuhan (CN); Xu ZHANG, Wuhan (CN); Maili LIU, Wuhan (CN)

(73) Assignee: Innovation Academy for Precision Measurement Science and Technology, Chinese Academy of Sciences, Wuhan (CN)

(21) Appl. No.: 19/464,491

(22) Filed: Jan. 29, 2026

Related U.S. Application Data
(63) Continuation of application No. PCT/CN2025/084391, filed on Mar. 24, 2025.

(30) Foreign Application Priority Data
Jan. 27, 2025 (CN) 202510125829.2

(51) Int. Cl. C12N 9/12; C12Q 1/686

(52) U.S. Cl. CPC C12N 9/1252; C12Q 1/686; C12Y 207/07007

(57) ABSTRACT

A Pfu DNA polymerase mutant and an application thereof in PCR are provided. Specifically, substitution occurs at an amino acid residue H680 and/or A684 in an amino acid sequence of a wild-type Pfu DNA polymerase or a wild-type Pfu DNA polymerase derivative. Compared with the unmutated polymerase, the resulting polymerase mutant exhibits a lower Kₘ value and a higher affinity for substrates, thereby enhancing the catalytic activity of the Pfu DNA polymerase.

### CROSS REFERENCE TO THE RELATED APPLICATIONS

This application is a continuation application of International Application No. PCT/CN2025/084391, filed on Mar. 24, 2025, which is based upon and claims priority to Chinese Patent Application No. 202510125829.2, filed on Jan. 27, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBWHZQ012_Sequence_Listing.xml, created on 01/22/2026, and is 46,878 bytes in size.

### TECHNICAL FIELD

The present invention relates to the technical field of PCR, and in particular to a Pfu DNA polymerase mutant and an application thereof in PCR.

### BACKGROUND

Polymerase Chain Reaction (PCR) technology is a core technique in molecular biology, possessing powerful functions, and can efficiently and rapidly amplify specific DNA or RNA sequences from different sources in vitro. Pfu DNA polymerase is found in the thermophilic archaeon Pyrococcus furiosus. The enzyme possesses both 5′-3′ polymerase activity and 3′-5′ exonuclease activity, allowing it to correct misincorporated bases during DNA replication in vivo, resulting in extremely high fidelity. However, with the development of new interdisciplinary fields such as synthetic biology, researchers increasingly pursue polymerases with different properties, such as higher affinity for substrates, the ability to amplify long fragments, compatibility with high-GC templates, or the capacity to extract DNA directly from crude extracts. Therefore, there is an urgent need to develop various Pfu DNA polymerase mutants to meet the growing demands of laboratory research and industrial production.

### SUMMARY

The purpose of the present invention is to address the above-mentioned problems in the prior art and to provide a Pfu DNA polymerase mutant with high affinity for substrates and high catalytic activity, and an application thereof in PCR.

To achieve the above purpose, the technical solution of the present invention is as follows:

The present invention provides a Pfu DNA polymerase mutant, and the Pfu DNA polymerase mutant is obtained by substitution at at least one amino acid residue selected from the group consisting of H680 and A684 in an amino acid sequence of a wild-type Pfu DNA polymerase or a wild-type Pfu DNA polymerase derivative.

Specifically, the substitution at the amino acid residue H680 is K, and the substitution at the amino acid residue A684 is Q.

Specifically, the amino acid sequences of the wild-type Pfu DNA polymerase and its H680K mutant, A684Q mutant, and H680K+A684Q mutant are shown as SEQ ID NO: 1 to SEQ ID NO: 4, respectively; and

the amino acid sequences of the wild-type Pfu DNA polymerase derivative and its H680K mutant, A684Q mutant, and H680K+A684Q mutant are shown as SEQ ID NO: 5 to SEQ ID NO: 8, respectively.

The present invention further provides a nucleotide sequence encoding the aforementioned Pfu DNA polymerase mutant, and the nucleotide sequences encoding the wild-type Pfu DNA polymerase and its H680K mutant, A684Q mutant, and H680K+A684Q mutant are shown as SEQ ID NO: 9 to SEQ ID NO: 12, respectively; and

the nucleotide sequences encoding the wild-type Pfu DNA polymerase derivative and its H680K mutant, A684Q mutant, and H680K+A684Q mutant are shown as SEQ ID NO: 13 to SEQ ID NO: 16, respectively.

The present invention further provides a recombinant vector including the aforementioned nucleotide sequence.

The present invention further provides a recombinant cell including the aforementioned nucleotide sequence or the aforementioned recombinant vector.

The present invention further provides an application of the aforementioned Pfu DNA polymerase mutant, the aforementioned nucleotide sequence, the aforementioned recombinant vector, or the aforementioned recombinant cell in the field of PCR.

Compared with the prior art, the present invention has the following advantages:

Compared with the unmutated Pfu DNA polymerase, the H680K mutant and the A684Q mutant in the present invention exhibit a lower Kₘ value, and a lower Kₘ value indicates a higher affinity for a substrate, thereby enhancing the catalytic activity of the Pfu DNA polymerase; in addition, compared with the unmutated Pfu DNA polymerase, the H680K mutant is obtained through mutating histidine at position 680 of the Pfu DNA polymerase to lysine, which enhances the surface electrostatic potential of the enzyme, thereby strengthening electrostatic interactions with the DNA template and further improving amplification specificity; and the A684Q mutant is obtained through mutating alanine at position 684 of the Pfu DNA polymerase to glutamine, the side chain of the glutamine is able to extend more deeply into the double-helical groove of the DNA template, thereby enhancing the affinity of the Pfu DNA polymerase for the DNA template, and reducing salt tolerance, thus improving compatibility with the high-GC DNA template.

### BRIEF DESCRIPTION OF THE DRAWINGS

FIG. 1 shows the PCR amplification results obtained by using an H680K mutant of a wild-type Pfu DNA polymerase derivative with upland cotton genomic DNA (gDNA) as a DNA template.

FIG. 2 shows a structural comparison diagram of a wild-type Pfu DNA polymerase derivative and an H680K mutant thereof.

FIG. 3 shows the PCR amplification results obtained by using an A684Q mutant of a wild-type Pfu DNA polymerase derivative with a pet16b-derived plasmid DNA as a DNA template under potassium salt concentrations ranging from 10 mM to 100 mM.

FIG. 4 shows the PCR amplification results obtained by using an A684Q mutant of a wild-type Pfu DNA polymerase derivative with Nipponbare rice gDNA as a DNA template under potassium salt concentrations of 50 mM and 100 mM.

FIG. 5 shows a structural comparison diagram of a wild-type Pfu DNA polymerase derivative and an A684Q mutant thereof.

### DETAILED DESCRIPTION OF THE EMBODIMENTS

For clearer illustration of the present invention, the present invention is further described below in conjunction with preferred embodiments and the accompanying drawings. Similar components in the drawings are denoted by the same reference numerals. A person skilled in the art should understand that the specific content described below is illustrative rather than limiting and shall not be construed as limiting the protection scope of the present invention.

The present invention provides a Pfu DNA polymerase mutant, which is obtained by substitution at at least one amino acid residue selected from the group consisting of H680 and A684 in an amino acid sequence of a wild-type Pfu DNA polymerase or a wild-type Pfu DNA polymerase derivative.

The substitution at the amino acid residue H680 is K, and the substitution at the amino acid residue A684 is Q.

The amino acid sequences of the wild-type Pfu DNA polymerase and its H680K mutant, A684Q mutant, and H680K+A684Q mutant are shown as SEQ ID NO: 1 to SEQ ID NO: 4, respectively; and

the wild-type Pfu DNA polymerase derivative is obtained through fusing a double-stranded DNA-binding protein sso7d to the C-terminus of the wild-type Pfu DNA polymerase, and the amino acid sequences of the wild-type Pfu DNA polymerase derivative and its H680K mutant, A684Q mutant, and H680K+A684Q mutant are shown as SEQ ID NO: 5 to SEQ ID NO: 8, respectively.

The present invention further provides a nucleotide sequence encoding the aforementioned Pfu DNA polymerase mutant, and the nucleotide sequences encoding the wild-type Pfu DNA polymerase and its H680K mutant, A684Q mutant, and H680K+A684Q mutant are shown as SEQ ID NO: 9 to SEQ ID NO: 12, respectively; and

the nucleotide sequences encoding the wild-type Pfu DNA polymerase derivative and its H680K mutant, A684Q mutant, and H680K+A684Q mutant are shown as SEQ ID NO: 13 to SEQ ID NO: 16, respectively.

The present invention further provides a recombinant vector including the aforementioned nucleotide sequence.

The present invention further provides a recombinant cell including the aforementioned nucleotide sequence or the aforementioned recombinant vector.

The present invention further provides an application of the aforementioned Pfu DNA polymerase mutant, the aforementioned nucleotide sequence, the aforementioned recombinant vector, or the aforementioned recombinant cell in the field of PCR.

The following explanation is provided through taking the H680K mutant, the A684Q mutant, and the H680K+A684Q mutant of the wild-type Pfu DNA polymerase derivative as examples. The main materials and instruments used in the present invention are shown in Table 1.

TABLE 1
Main Materials and Instruments

| Reagent/Instrument Name | Model | Manufacturer |
|---|---|---|
| T4 DNA ligase | — | Sigma Aldrich Company |
| Constant Temperature | DNP-9162 | Shanghai Jinghong |
| Incubator | | |
| Protein Purification | NGC Quest | Bio-Rad Company |
| Instrument | 10plus | |
| PCR Instrument | C1000 Touch | Bio-Rad Company |
| PH Meter | FiveEasy Plus | METTLER Company |
| Electrophoresis Apparatus | 164-5050 | Bio-Rad Company |
| Benchtop Centrifuge | FRESCO17 | Thermo Scientific |
| | | Company |
| NanoDrop ™ One/One^{C} | 8400-317400 | Thermo Scientific |
| Ultramicro UV | | Company |
| Spectrophotometer | | |

### Embodiment 1 Enzymatic Kinetic Analysis of Wild-Type Pfu DNA Polymerase Derivative Mutants

Method for Enzymatic Kinetic Analysis: The hairpin DNA is pretreated through heating at 98 °C for 5 min followed by annealing on ice for 30 min; the hairpin DNA at concentrations of 0.1-2 M is used as the substrate template and mixed with dNTP and 1 nM of the wild-type Pfu DNA polymerase derivative or its mutant, and DNA synthesis is initiated through adding MgCl₂ at 72 °C; the reaction buffer includes 25 mM Tris-HCl (pH 8.8), 10 mM KCl, 2.5 mM MgCl₂, 200 M dNTP, 0.05% Triton X-100, and 1× EvaGreen; and fluorescence curves are plotted, with each curve obtained by subtracting the fluorescence value from the background of the corresponding reaction, and the first derivative of the fluorescence curves is calculated to obtain the initial reaction rates for each reaction under different template amounts. GraphPad Prism 8.0 software is used to fit the Michaelis-Menten kinetic equation to obtain values of k_{cat} and Kₘ. Three biological replicates are set for each group. The base sequence of the hairpin DNA is 5′-CCAGCATTATGAAAGTGACACGTGCACCATTGGTGCACGTG-3′ (SEQ ID NO: 17). The enzymatic kinetic analysis results of the wild-type Pfu DNA polymerase derivative and its A684Q, H680K, and A684Q+H680K mutants are shown in Table 2.

TABLE 2
Enzymatic Kinetic Analysis Results of Wild-Type
Pfu DNA Polymerase Derivative and Mutants Thereof

| Enzyme Type | k_{cat} (s⁻¹) | Kₘ (μM) |
|---|---|---|
| Wild-Type Pfu | 190.70 ± 11.07 | 0.45 ± 0.07 |
| DNA | | |
| Polymerase | | |
| Derivative | | |
| H680K | 173.28 ± 5.36 | 0.31 ± 0.06 |
| A684Q | 194.90 ± 2.361 | 0.34 ± 0.03 |
| A684Q + H680K | 192.30 ± 6.63 | 0.23 ± 0.03 |

As can be seen from Table 2, the wild-type Pfu DNA polymerase derivative has a Kₘ value of 0.45±0.07, whereas the Kₘ values of the A684Q mutant and the H680K mutant are 0.34±0.03 and 0.31±0.06, respectively. The results show that the A684Q mutant, the H680K mutant, and the A684Q+H680K mutant all exhibit lower Kₘ values. A lower Kₘ value indicates a stronger binding affinity to the substrate. Among these mutants, the A684Q+H680K mutant exhibits the lowest Kₘ value, indicating that the A684Q+H680K mutant has the strongest substrate-binding affinity.

### Embodiment 2 Amplification Performance Analysis of Wild-Type Pfu DNA Polymerase Derivative Mutants

PCR amplification is performed on different DNA templates using the A684Q mutant and the H680K mutant of the wild-type Pfu DNA polymerase derivative. The PCR amplification system includes: dNTP at 200 μmol/L, the wild-type Pfu DNA polymerase derivative mutant at 40 U/mL, hsp28 at 200 pg/μL, Tris at 120 mmol/L, KCl at 10 mmol/L, (NH₄)₂SO₄ at 6 mmol/L, MgCl₂ at 2 mmol/L, Triton X-100 at 0.100 (w/v), and BSA at 0.001% (w/v); and the pH ranges from 8.3 to 9.2. PCR amplification method includes the step of adding a DNA template, a forward primer, and a reverse primer to the foregoing PCR amplification system, and performing a PCR reaction using a PCR instrument. The PCR amplification program is shown in Table 3. The DNA templates used for the A684Q mutant include a pet16b-derived plasmid DNA with a target length of 10 kbp and Nipponbare rice gDNA with a target length of 417 kbp, wherein the Nipponbare rice gDNA is a high-GC template, and the PCR amplification is performed under different potassium salt concentrations ranging from 10 mM to 100 mM; and the DNA template used for the H680K mutant is upland cotton gDNA with a target length of 1.3 kbp. The corresponding forward primers and reverse primers are shown in Table 4.

TABLE 3
PCR Amplification Program

| | | | | |
|---|---|---|---|---|
| 98 °C, | 98 °C, | 55 °C, | 72 °C, | 72 °C, |
| 1 min | 15 s | 15 s | 30 s/kb | 10 min |

18-50 Cycles

TABLE 4
Forward Primers and Reverse Primers

| Enzyme | DNA | | |
|---|---|---|---|
| Type | Template | Primer | Base Sequence |
| A684Q | pet16b- | Forward | 5′-ATCCTGGACGTGGATTACATCACC-3′ |
| | Derived | Primer | (SEQ ID NO: 18) |
| | Plasmid | Reverse | 5′-CATATGACGACCTTCGATATGGCC-3′ |
| | DNA | Primer | (SEQ ID NO: 19) |
| | Nipponbare | Forward | 5′-ATGGCGTCCTACGACAAGGCC-3′ |
| | Rice | Primer | (SEQ ID NO: 20) |
| | gDNA | Reverse | 5′-TTATTTGCTTGCATCTTGGGGATCTTGATTCTC-3′ |
| | | Primer | (SEQ ID NO: 21) |
| H680K | Upland | Forward | 5′-GGTACCGAGATACAGAAGTTTTAG-3′ |
| | Cotton | Primer | (SEQ ID NO: 22) |
| | gDNA | Reverse | 5′-GTCGACCATCTAGAACAGGAGTGAC-3′ |
| | | Primer | (SEQ ID NO: 23) |

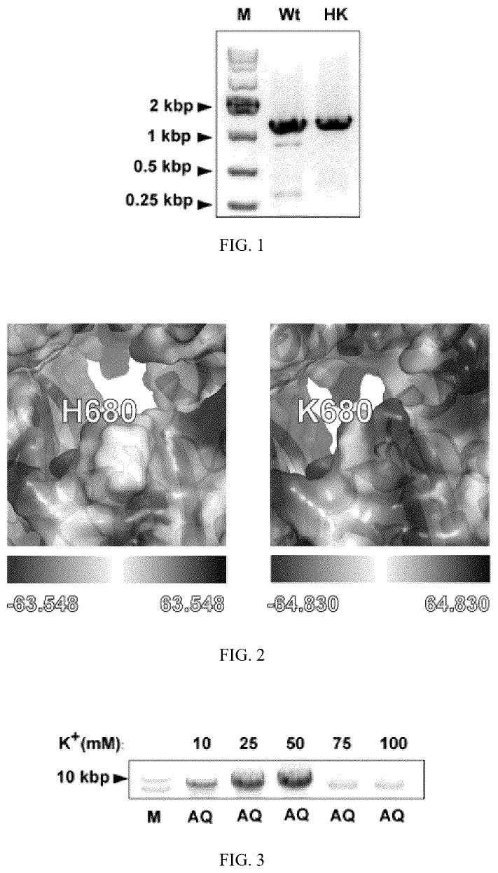

The PCR amplification result obtained using the H680K mutant with the upland cotton gDNA as the DNA template is shown in FIG. 1. In the figure, M represents the corresponding DNA template, WT represents the wild-type Pfu DNA polymerase derivative, and HK represents the H680K mutant of the wild-type Pfu DNA polymerase derivative. It can be seen that the H680K mutant significantly improves the amplification specificity of the polymerase. Structural analysis of the wild-type Pfu DNA polymerase derivative and its H680K mutant is performed using AlphaFold3 software, and the results are shown in FIG. 2. In the figure, H680 represents the wild-type Pfu DNA polymerase derivative, and K680 represents the H680K mutant. It can be seen that the H680K mutant increases the surface charge of the wild-type Pfu DNA polymerase derivative.

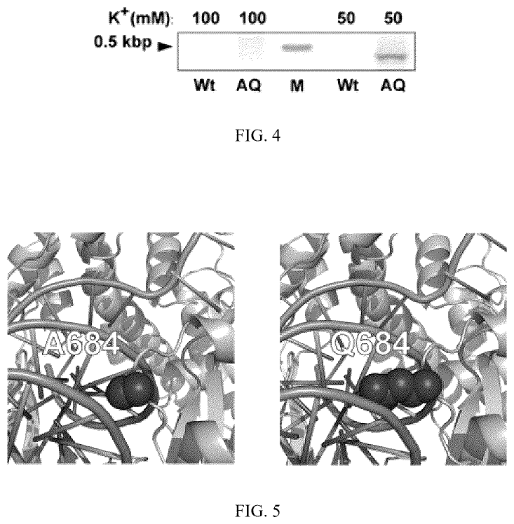

The PCR amplification result obtained using the A684Q mutant with pet16b-derived plasmid DNA as the DNA template is shown in FIG. 3. In the figure, M represents the corresponding DNA template, and AQ represents the A684Q mutant of the Pfu DNA polymerase. It can be seen that the salt tolerance of the A684Q mutant is 50 mM, whereas the wild-type Pfu DNA polymerase derivative generally has a salt tolerance of 100 mM, indicating that the A684Q mutant has lower salt tolerance. The PCR amplification result obtained using the A684Q mutant with Nipponbare rice gDNA as the DNA template is shown in FIG. 4. In the figure, M represents the corresponding DNA template, WT represents the wild-type Pfu DNA polymerase derivative, and AQ represents the A684Q mutant of the wild-type Pfu DNA polymerase derivative. It can be seen that the A684Q mutant exhibits higher amplification performance for high-GC DNA templates at a salt concentration of 50 mM. Structural analysis of the Pfu DNA polymerase and its A684Q mutant is performed using AlphaFold3 software, and the results are shown in FIG. 5. In the figure, A684 represents the Pfu DNA polymerase, and Q684 represents the A684Q mutant. It can be seen that the glutamine side chain in the A684Q mutant can insert more deeply into the double-helix groove of the DNA template, resulting in tighter binding.

### Embodiment 3 Fidelity Analysis of Wild-Type Pfu DNA Polymerase Derivative Mutants

Fidelity Analysis: The linearized pUC19 plasmid vector carrying the lacZ gene is amplified, and the amplification products are subjected to agarose gel electrophoresis. The gel of the target band of the linearized vector is recovered, the gel-recovered product is ligated using T4 DNA ligase, and the ligation product is then transformed into the α-complementation Escherichia coli (E. coli) host strain Turbo. Aliquots of the infected cells are plated onto LB plates including top agar with 1 mg/mL X-gal and 1.5 mM IPTG and incubated overnight at 37 °C, then standard blue-white colony screening is performed, the numbers of blue and white colonies are counted, and the apparent mutation frequency is calculated as: apparent mutation frequency=(white colonies/total colonies)*100. Three biological replicates are set for each group. The fidelity analysis results are shown in Table 5.

TABLE 5
Fidelity Analysis Results of Wild-Type Pfu DNA Polymerase
Derivative and Its A684Q and H680K Mutants

| | Apparent Mutation |
|---|---|
| Enzyme Type | Frequency (%) |
| Wild-Type Pfu DNA | 0.41 ± 0.04 |
| Polymerase | |
| Derivative | |
| H680K | 0.35 ± 0.05 |
| A684Q | 0.43 ± 0.03 |

As shown in Table 5, the apparent mutation frequency of the wild-type Pfu DNA polymerase derivative is 0.41±0.04%, while that of the A684Q mutant is 0.35±0.05%, indicating that the fidelity of the A684Q mutant is essentially comparable to that of the wild-type Pfu DNA polymerase derivative. The H680K mutant exhibits a slightly lower error rate of 0.35±0.05%, indicating that the H680K mutant has improved fidelity compared with the unmutated wild-type Pfu DNA polymerase derivative.

In summary, the two mutants of the wild-type Pfu DNA polymerase derivative in the present invention maintain or exhibit higher fidelity in PCR assays, while improving the Kₘ value of the wild-type Pfu DNA polymerase derivative and enhancing the amplification performance of the enzyme under different conditions. These mutants can be applied in various cloning, functional analysis, and sequencing applications.

### Embodiment 4

The nucleotide sequence encoding the Pfu DNA polymerase derivative is constructed into an E. coli expression vector to generate recombinant E. coli cells. During PCR, a 6-minute pre-denaturation step lyses the recombinant E. coli cells, and PCR amplification is used to screen an NKK-saturated mutation library at positions H680 and A684, resulting in the identification of two beneficial mutants of the Pfu DNA polymerase derivative, namely the H680K mutant and the A684Q mutant.

What is claimed is:

1. A Pfu DNA polymerase mutant, wherein the Pfu DNA polymerase mutant is obtained by substitution at at least one amino acid residue selected from the group consisting of H680 and A684 in an amino acid sequence of a wild-type Pfu DNA polymerase or a wild-type Pfu DNA polymerase derivative.

2. The Pfu DNA polymerase mutant according to claim 1, wherein a substitution at the amino acid residue H680 is K, and a substitution at the amino acid residue A684 is Q.

3. The Pfu DNA polymerase mutant according to claim 2, wherein
the amino acid sequences of the wild-type Pfu DNA polymerase and its H680K mutant, A684Q mutant, and H680K+A684Q mutant are shown as SEQ ID NO: 1 to SEQ ID NO: 4, respectively; and
the amino acid sequences of the wild-type Pfu DNA polymerase derivative and its H680K mutant, A684Q mutant, and H680K+A684Q mutant are shown as SEQ ID NO: 5 to SEQ ID NO: 8, respectively.

4. A nucleotide sequence encoding the Pfu DNA polymerase mutant according to claim 3, wherein
the nucleotide sequences encoding the wild-type Pfu DNA polymerase and its H680K mutant, A684Q mutant, and H680K+A684Q mutant are shown as SEQ ID NO: 9 to SEQ ID NO: 12, respectively; and
the nucleotide sequences encoding the wild-type Pfu DNA polymerase derivative and its H680K mutant, A684Q mutant, and H680K+A684Q mutant are shown as SEQ ID NO: 13 to SEQ ID NO: 16, respectively.

5. A recombinant vector comprising the nucleotide sequence according to claim 4.

6. A recombinant cell comprising the nucleotide sequence according to claim 4 or a recombinant vector comprising the nucleotide sequence.

7. A PCR method, comprising using the Pfu DNA polymerase mutant according to claim 1.

8. The PCR method according to claim 7, wherein in the Pfu DNA polymerase mutant, a substitution at the amino acid residue H680 is K, and a substitution at the amino acid residue A684 is Q.

9. A PCR method, comprising using the Pfu DNA polymerase mutant according to claim 3, a nucleotide sequence, a recombinant vector, or a recombinant cell, wherein the nucleotide sequence encodes the Pfu DNA polymerase mutant, the nucleotide sequences encoding the wild-type Pfu DNA polymerase and its H680K mutant, A684Q mutant, and H680K+A684Q mutant are shown as SEQ ID NO: 9 to SEQ ID NO: 12, respectively; and
the nucleotide sequences encoding the wild-type Pfu DNA polymerase derivative and its H680K mutant, A684Q mutant, and H680K+A684Q mutant are shown as SEQ ID NO: 13 to SEQ ID NO: 16, respectively;
the recombinant vector comprises the nucleotide sequence;
the recombinant cell comprises the nucleotide sequence or the recombinant vector.


SEQUENCE LISTING (23 sequences)

SEQ ID NO: 1 - protein, 775 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 2 - protein, 775 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLKEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 3 - protein, 775 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKQIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 4 - protein, 775 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLKEYKQIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 5 - protein, 844 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 6 - protein, 844 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLKEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 7 - protein, 844 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKQIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 8 - protein, 844 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLKEYKQIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 9 - DNA, 2328 bp, synthetic construct
  source 1..2328: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgcacgaatacaaagccatcggtccgcatgttgcggtagcgaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagctaa

SEQ ID NO: 10 - DNA, 2328 bp, synthetic construct
  source 1..2328: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgaaagaatacaaagccatcggtccgcacgttgcggtagcgaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagctaa

SEQ ID NO: 11 - DNA, 2328 bp, synthetic construct
  source 1..2328: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgcacgaatacaaacagatcggtccgcatgttgcggtagccaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagctaa

SEQ ID NO: 12 - DNA, 2328 bp, synthetic construct
  source 1..2328: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgaaagaatacaaacagatcggtccgcatgttgcggtagccaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagctaa

SEQ ID NO: 13 - DNA, 2535 bp, synthetic construct
  source 1..2535: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgcacgaatacaaagccatcggtccgcatgttgcggtagcgaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagcggtaccggcggcggtggcgcaaccgttaaatttaaatacaaaggtgaagaaaaagaagttgacatcagcaaaattaagaaagtgtggcgcgtgggtaaaatgatctcctttacctacgacgaaggcggcggcaaaaccggtcgcggtgcggtgtccgaaaaagatgcgccgaaagaactgctgcagatgctggaaaaacagaaaaaataa

SEQ ID NO: 14 - DNA, 2535 bp, synthetic construct
  source 1..2535: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgaaagaatacaaagccatcggtccgcacgttgcggtagcgaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagcggtaccggcggcggtggcgcaaccgttaaatttaaatacaaaggtgaagaaaaagaagttgacatcagcaaaattaagaaagtgtggcgcgtgggtaaaatgatctcctttacctacgacgaaggcggcggcaaaaccggtcgcggtgcggtgtccgaaaaagatgcgccgaaagaactgctgcagatgctggaaaaacagaaaaaataa

SEQ ID NO: 15 - DNA, 2535 bp, synthetic construct
  source 1..2535: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgcacgaatacaaacagatcggtccgcatgttgcggtagccaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagcggtaccggcggcggtggcgcaaccgttaaatttaaatacaaaggtgaagaaaaagaagttgacatcagcaaaattaagaaagtgtggcgcgtgggtaaaatgatctcctttacctacgacgaaggcggcggcaaaaccggtcgcggtgcggtgtccgaaaaagatgcgccgaaagaactgctgcagatgctggaaaaacagaaaaaataa

SEQ ID NO: 16 - DNA, 2535 bp, synthetic construct
  source 1..2535: mol_type = other DNA
atgatcctggacgtggattacatcaccgaagaaggcaaaccggtgatccgcctgttcaaaaaagaaaacggtaaattcaaaatcgaacacgaccgtaccttccgtccgtacatctacgcgctgctgcgtgatgattccaaaatcgaagaagtgaagaaaatcaccggcgaacgccatggcaaaatcgtgcgtattgtcgatgttgaaaaagtggagaaaaaattcctgggtaaaccaatcaccgtttggaaactgtacctggaacatccgcaggacgtgccgaccatccgtgaaaaagtgcgcgaacacccggcggtagtggatatcttcgagtacgacattccgttcgccaaacgttatctgatcgataaaggcctgatcccgatggaaggcgaagaagagctgaaaattctggccttcgacatcgaaaccctgtatcacgaaggtgaggaattcggtaaaggcccgatcatcatgatctcttacgccgacgaaaacgaagcgaaagtgatcacctggaaaaacatcgatctgccgtacgtggaagtggtttcctccgagcgcgaaatgatcaaacgtttcctgcgcattatccgtgaaaaagacccggatatcattgtaacctacaacggcgatagcttcgatttcccatatctggcgaaacgtgctgaaaaactgggtattaaactgaccattggtcgtgacggctctgaaccgaaaatgcagcgtatcggtgatatgaccgcggttgaagtcaaaggccgcatccacttcgatctgtatcacgtgatcacccgtaccattaacctgccgacctatacgctggaagcggtttatgaagcgatcttcggcaaaccgaaagaaaaagtgtacgcggatgaaatcgcaaaagcatgggaatccggcgaaaacctggaacgcgttgcgaaatattctatggaagacgcgaaagctacctatgagctgggcaaagaattcctgccgatggaaatccagctgtcccgcctggtgggccaaccgctgtgggacgtttcccgcagctccaccggtaatctggttgaatggtttctgctgcgcaaagcatacgaacgtaatgaggtggccccgaacaaaccgtccgaagaagaataccagcgtcgcctgcgcgaatcctacaccggcggtttcgtgaaagaaccggagaaaggcctgtgggagaacattgtatacctggacttccgcgcgttgtacccgtctattattatcacccacaacgtgagcccggacaccttgaacctggagggctgcaaaaactacgacatcgctccgcaggtgggccataaattctgtaaagacatccctggtttcatcccaagcctgcttggccacctgctggaagagcgtcagaaaatcaaaacgaaaatgaaagaaacccaggacccgatcgaaaaaatcctgctggattaccgtcagaaagcgatcaagctgctggcgaactccttctacggttattatggctacgcgaaagcgcgttggtactgtaaagaatgcgctgaaagcgtgaccgcctggggccgtaaatacattgaactggtgtggaaagaactggaagaaaaattcggctttaaagtgctgtatatcgacaccgatggtctgtacgcgacgattccaggcggcgaatctgaagaaattaaaaagaaagcgctggaatttgttaaatatattaacagcaaactgccgggcctgctggaattagaatacgaaggcttctacaaacgcggtttcttcgttaccaaaaaacgctacgctgtgattgatgaagagggcaaagttatcacccgcggcctggaaattgttcgccgtgactggtccgaaatcgctaaagaaacccaggcgcgtgtgctggaaaccattctgaaacacggcgacgttgaagaagcggttcgtatcgtgaaagaagttatccagaaactggcgaactacgaaattccgccggaaaaactcgccatctacgaacagatcacgcgtccgctgaaagaatacaaacagatcggtccgcacgttgcggtagcgaaaaaactggctgctaaaggtgttaaaatcaaaccgggcatggttatcggttatatcgttctccgtggtgatggcccgatcagcaaccgcgcgatcctggcggaagaatacgacccgaaaaagcataaatacgatgcggaatactacatcgaaaaccaggttctgccggcagtgctgcgcatcctggaaggttttggctaccgtaaagaagacctgcgttatcagaaaacccgtcaggttggtctcaccagctggctgaacattaaaaagagcggtaccggcggcggtggcgcaaccgttaaatttaaatacaaaggtgaagaaaaagaagttgacatcagcaaaattaagaaagtgtggcgcgtgggtaaaatgatctcctttacctacgacgaaggcggcggcaaaaccggtcgcggtgcggtgtccgaaaaagatgcgccgaaagaactgctgcagatgctggaaaaacagaaaaaataa

SEQ ID NO: 17 - DNA, 41 bp, unidentified
  source 1..41: mol_type = other DNA
ccagcattatgaaagtgacacgtgcaccattggtgcacgtg

SEQ ID NO: 18 - DNA, 24 bp, unidentified
  source 1..24: mol_type = other DNA
atcctggacgtggattacatcacc

SEQ ID NO: 19 - DNA, 24 bp, unidentified
  source 1..24: mol_type = other DNA
catatgacgaccttcgatatggcc

SEQ ID NO: 20 - DNA, 21 bp, unidentified
  source 1..21: mol_type = other DNA
atggcgtcctacgacaaggcc

SEQ ID NO: 21 - DNA, 33 bp, unidentified
  source 1..33: mol_type = other DNA
ttatttgcttgcatcttggggatcttgattctc

SEQ ID NO: 22 - DNA, 24 bp, unidentified
  source 1..24: mol_type = other DNA
ggtaccgagatacagaagttttag

SEQ ID NO: 23 - DNA, 25 bp, unidentified
  source 1..25: mol_type = other DNA
gtcgaccatctagaacaggagtgac